SEQUENCE LISTING (4 sequences)

SEQ ID NO: 1 - DNA, 22 bp, Artificial
  primer
atgaaggttttcgagagatgcg

SEQ ID NO: 2 - DNA, 27 bp, Artificial
  primer
ggggtaccaacaccacaaccttgaacg

SEQ ID NO: 3 - DNA, 19 bp, Artificial
  primer
gcagattgtactgagagtg

SEQ ID NO: 4 - DNA, 19 bp, Artificial
  primer
gcggataacaatttcacac

(19) United States
(12) Patent Application Publication
(10) Pub. No.: US 2009/0155881 A1
(43) Pub. Date: Jun. 18, 2009

(54) CELL-FREE PROTEIN SYNTHESIS METHOD AND CELL-FREE PROTEIN SYNTHESIS REACTION SOLUTION USING ADENOSINE 3',5'-BISPHOSPHATE

(76) Inventors: Masamitsu Shikata, Kyoto-shi (JP); Masaaki Ito, Kyoto-shi (JP); Toru Ezure, Kyoto-shi (JP); Takashi Suzuki, Kyoto-shi (JP)

Correspondence Address: Cheng Law Group, PLLC, 1100 17th Street, N.W., Suite 503, Washington, DC, 20036, US

(21) Appl. No.: 12/323,233

(22) Filed: Nov. 25, 2008

(30) Foreign Application Priority Data
Dec. 13, 2007 (JP) 2007-321946

(51) Int. Cl. C12N 9/12

(52) U.S. Cl. 435/194

(57) ABSTRACT

The present invention provides a method of conducting cell-free protein synthesis by conveniently suppressing mRNA degradation, and a reaction solution enabling cell-free protein synthesis by conveniently suppressing mRNA degradation. A cell-free protein synthesis method using a cell-free protein synthesis reaction solution containing at least an extract liquid derived from a living cell, a potassium salt, a magnesium salt, adenosine triphosphate, guanosine triphosphate, creatine phosphate, creatine kinase, amino acid, a tRNA, an mRNA, a buffer, and adenosine 3′,5′-bisphosphate.

### BACKGROUND OF THE INVENTION

1. Field of the Invention

The present invention relates to a cell-free protein synthesis method. More specifically, the present invention relates to a method of conducting cell-free protein synthesis while suppressing degradation of an mRNA. Concretely, the present invention relates to a cell-free protein synthesis method and a cell-free protein synthesis reaction solution using adenosine 3′,5′-bisphosphate.

2. Disclosure of the Related Art

Since there exist a plurality of ribonucleases involved in an mRNA metabolism in a cell, a cell extract liquid has ribonuclease activity. Therefore, in cell-free protein synthesis using a cell extract liquid, degradation of an mRNA which is to be a template, by a ribonuclease is problematic.

For this reason, in the art of cell-free protein synthesis, cell-free protein synthesis is often conducted while using a commercially available ribonuclease inhibitor (derived from a human placenta or from a hog liver) for the purpose of overcoming the problem of degradation of an mRNA.

Here, an mRNA which is present in a cell is generally composed of a CAP structure, a 5′-untranslated region, a translated region, a 3′-untranslated region and a poly-A tail from the 5′ side. An mRNA having completed translation is rapidly degraded. It is known that at this time, when an mRNA is degraded to a certain length as a result of degradation of the polyA tail existing in the 3′ end, the mRNA is then degraded at a stroke by a 5′-exonuclease from the 5′ side triggered by a CAP removal reaction (Non-patent Document 1: Microbiol Rev. March 1996; 60(1): 233-49).

In view of the above, in the field of cell-free protein synthesis, a method of suppressing degradation of an mRNA which is to be a template from its end is reported. For example, it is known that adding a CAP structure is useful. Also, a method of looping the 5′-end and the 3′-end of an mRNA, thereby improving the synthesis amount of a protein is reported (Non-patent Document 2: Current Opinion in Biotechnology 1998, 9:534-548).

### SUMMARY OF THE INVENTION

The measure of adding a CAP structure as described above faces problems that the cost increases, and a pre-treatment is troublesome. The measure of looping the 5′-end and the 3′-end (see the above Non-patent Document 2) requires labor because it is necessary to design and prepare a template DNA in such a manner. Therefore, any of these is not used at present in a general cell-free protein synthesis technique, or not used as a practical technique even if it is used. Therefore, it is often the case that cell-free protein synthesis is conducted while only a ribonuclease inhibitor is added.

However, ribonuclease inhibitors conventionally used in a cell-free protein technique inhibit the effect of an endonuclease that is involved in degradation of an mRNA from its midpoint concretely like RNase A. On the other hand, such a ribonuclease inhibitor does not have an effect of inhibiting an exonuclease that is involved in degradation from the 5′-end or from the 3′-end. Therefore, in the reaction solution as a whole, suppression of RNA degradation is not effectively achieved.

In light of the above, it is an object of the present invention to provide a method of conducting cell-free protein synthesis by conveniently suppressing mRNA degradation, and a reaction solution enabling cell-free protein synthesis by conveniently suppressing mRNA degradation.

As a result of diligent efforts, inventors of the present invention found an mRNA degradation suppressing effect by adenosine 3′,5′-bisphosphate in a cell-free translation system, and accomplished the present invention.

The present invention includes the following aspects.

(1) A cell-free protein synthesis method using a cell-free protein synthesis reaction solution containing at least an extract liquid derived from a living cell, a potassium salt, a magnesium salt, adenosine triphosphate, guanosine triphosphate, creatine phosphate, creatine kinase, amino acid, a tRNA, an mRNA, a buffer, and adenosine 3′,5′-bisphosphate.

Adenosine 3′,5′-bisphosphate is also referred to as adenosine 3′,5′-diphosphate or 3′-phosphoadenosine-5′-phosphate.

By containing adenosine 3′,5′-bisphosphate in the cell-free protein synthesis reaction solution, it is possible to suppress degradation of an mRNA.

The cell-free protein synthesis method wherein the adenosine 3′,5′-bisphosphate is contained at a concentration of 1 to 30 mM in the cell-free protein synthesis reaction solution.

By setting the concentration of adenosine 3′,5′-bisphosphate within the above range, a more effective mRNA degradation suppressing effect is achieved.

(2) The cell-free protein synthesis method as described in (1), wherein the cell-free protein synthesis reaction solution further contains lithium ion Li⁺.

By further containing lithium ion in the cell-free protein synthesis reaction solution, the mRNA degradation suppressing effect by pAp can be achieved more effectively with higher sustention or with lower cost.

(3) The cell-free protein synthesis method as described in (1) or (2), wherein while the cell-free protein synthesis reaction is sustained, adenosine 3′,5′-bisphosphate is supplemented to the cell-free protein synthesis reaction solution.

By supplementing adenosine 3′,5′-bisphosphate to the cell-free protein synthesis reaction solution, the mRNA degradation suppressing effect is achieved more effectively with higher sustention.

(4) The cell-free protein synthesis method as described in any one of (1) to (3), wherein the living cell is an insect culture cell.

(5) A cell-free protein synthesis reaction solution containing at least an extract liquid derived from a living cell, a potassium salt, a magnesium salt, adenosine triphosphate, guanosine triphosphate, creatine phosphate, creatine kinase, amino acid, a tRNA, an mRNA, a buffer, and adenosine 3′,5′-bisphosphate.

(6) The cell-free protein synthesis reaction solution as described in (5), further containing lithium ion Li⁺.

According to the present invention, it is possible to provide a method of conducting cell-free protein synthesis by conveniently suppressing mRNA degradation, and a reaction solution enabling cell-free protein synthesis by conveniently suppressing mRNA degradation.

### BRIEF DESCRIPTION OF THE DRAWINGS

FIG. 1 is an electrophoresis image detecting RNAs contained in reaction solutions after subjecting Reaction Solutions 1 to 3 (lanes 1 to 3, respectively) not added with pAp and Reaction Solution 4 (lane 4) added with pAp, to a translation reaction (after 0, 60, and 120 minutes) in Example 1;

FIG. 2 is an electrophoresis image detecting RNAs contained in reaction solutions after subjecting Reaction Solution 4 (lane 4) added with 5 mM of pAp, Reaction Solution 5 (lane 5) added with 10 mM of pAp, and Reaction Solution 6 (lane 6) added with 5 mM of pAp with further supplementation, to a translation reaction (after 0, 120, 180, and 240 minutes) in Example 2; and

FIG. 3 is an electrophoresis image detecting RNAs contained in reaction solutions after subjecting Reaction Solution 4 (lane 4) added with 5 mM of pAp, Reaction Solution 7 (lane 7) added with 50 mM of LiCl, and Reaction Solution 8 (lane 8) added with 5 mM of pAp and 50 mM of LiCl, to a translation reaction (after 0, 180, and 240 minutes) in Example 3.

### DETAILED DESCRIPTION OF THE INVENTION

In the present invention, protein synthesis is conducted by containing adenosine 3′,5′-bisphosphate (hereinafter, also referred to as “pAp”) in a cell-free protein synthesis system. The term “protein” used herein embraces oligopeptide, and polypeptide.

As for ingredients that constitute a reaction solution for cell-free protein synthesis, basically ingredients used in a known reaction solution for cell-free protein synthesis may be recited without any particular limitation except that pAp is an essential ingredient, and an RNase inhibitor is not an essential ingredient. As for other ingredients than pAp, the one containing at least an extract liquid derived from a living cell, a potassium salt, a magnesium salt, adenosine triphosphate, guanosine triphosphate, creatine phosphate, creatine kinase, amino acid, a tRNA, an mRNA, and a buffer in water is usually used.

Among these, as the extract liquid derived from a living cell, those known in the art may be used without any limitation, however, it is particularly preferred to use an extract liquid derived from an insect culture cell. In particular, it is preferred to use High Five (available from Invitrogen) which is a cell derived from an egg cell of Trichoplusia ni or Sf21 (available from Invitrogen) which is a cell derived from an ovary cell of Spodoptera frugiperda as an insect cell because they have high protein synthesizing ability and can be cultured in a serum-free medium.

A preparation method of an insect cell extract liquid preferably used in the cell-free protein synthesis system according to the present invention is not particularly limited, for example, the method described in Japanese Patent Laid-open Publication No. 2004-215651, namely the method in which insect cells suspended in a liquid for extraction are rapidly frozen, and then the frozen insect cells are ground, followed by extraction may be used. This method is desirably used in that components necessary for cell-free protein synthesis can be taken out of the cells without being broken because cell grinding is executed in a gentle condition, in that contamination of RNase for example, from a tool being used can be prevented, and in that import of a translation reaction inhibiting substance that is concerned in cell grinding using a reagent such as surfactant is avoided.

Specifically, an insect cell extract liquid may be prepared in the form of an aqueous solution having a protein concentration of 1 mg/mL to 200 mg/mL, preferably 10 mg/mL to 100 mg/mL, containing 10 mM to 500 mM, preferably 50 mM to 300 mM of potassium acetate, 0.1 mM to 10 mM, preferably 0.5 mM to 5 mM of magnesium acetate, 1 μM to 50 mM, preferably 0.01 mM to 5 mM of PMSF (phenyl methyl sulfonyl fluoride), and 5 mM to 200 mM, preferably 10 mM to 100 mM of HEPES (2-[4-(2-hydroxyethyl)-1-piperazinyl]ethane sulfonic acid)-KOH (pH 4 to 10, preferably 6.5 to 8.5), which is preferably subjected to a nuclease treatment. In addition to the above, 0.1 mM to 10 mM, preferably 0.5 mM to 5 mM of dithiothreitol (DTT) may be contained.

In addition, a reaction solution for cell-free protein synthesis is preferably prepared to contain 10(v/v) % to 80(v/v) %, particularly 30(v/v) % to 60(v/v) % of such an insect cell extract liquid. That is, the reaction solution is prepared so that the solution as a whole contains an extract derived from an insect cell in an amount of preferably 0.1 mg/mL to 160 mg/mL, more preferably 3 mg/mL to 60 mg/mL by protein concentration. This is because when the content of the extract is less than 0.1 mg/mL or more than 160 mg/mL by protein concentration, the synthesis speed of an objective protein may be deteriorated.

The potassium salt, magnesium salt, adenosine triphosphate (ATP), guanosine triphosphate (GTP), creatine phosphate, creatine kinase, an amino acid component, an RNase inhibitor, a tRNA, a foreign mRNA, and a buffer for use in a reaction solution as ingredients other than the above extract liquid may be appropriately selected by a person skilled in the art. For example, it is preferred that the reaction solution is realized by an aqueous solution containing 50 mM to 150 mM of potassium acetate, 0.5 mM to 3 mM of magnesium acetate, 0.1 mM to 5 mM of ATP, 0.05 mM to 5 mM of GTP, 10 mM to 100 mM of creatine phosphate, 10 μg/mL to 500 μg/mL of creatine kinase, 10 μM to 200 μM of an amino acid component, 10 μg/mL to 500 μg/mL of a tRNA, and 20 μg/mL to 100 μg/mL of a foreign mRNA, and 10 mM to 50 mM of HEPES-KOH (pH 6.5 to 8.5).

In addition to the above, the reaction solution may also be realized by an aqueous solution containing dithiothreitol (DTT) (for example, 0.2 mM to 5 mM), and glycol ether diamine tetraacetic acid (EGTA) (for example, 0.1 mM to 10 mM) in addition to the above.

In the cell-free protein synthesis reaction solution of the present invention, pAp is further contained in addition to the above ingredients. By containing pAp in a reaction solution, it is possible to suppress degradation of an mRNA. The mRNA degradation suppressing effect by pAp is excellent in that, even in such a specific environment where a large quantity of an mRNA is present as in the cell-free protein synthesis reaction solution, such a large quantity of an mRNA can be effectively maintained.

The addition amount of pAp is not particularly limited, and for example, pAp may be contained in a reaction solution in a final concentration of, for example, 1 to 30 mM, preferably 5 to 20 mM. At a concentration lower than the above concentration, the effect of suppressing degradation of an mRNA is difficult to appear.

During the reaction, pAp tends to be reduced. This is partly attributable to the fact that it is metabolized by a component contained in the cell extract liquid.

In view of this, according to the present invention, pAp may be supplemented during sustention of reaction after starting the cell-free protein synthesis reaction. This makes it possible to obtain the effect of suppressing degradation of an mRNA more effectively with higher sustention.

The timing of supplementation, and the supplementation amount are not particularly limited. The timing of supplementation maybe, for example, after 30 to 60 minutes from starting of the reaction. In particular, when pAp is added in a final concentration of 5 mM, it is preferred to supplement pAp after 30 to 60 minutes from starting of the reaction. The supplementation amount may be set so that a final concentration of supplemented pAp excluding pAp that is present at the start of the reaction and is possibly remaining in the reaction solution at the time of supplementation, is 5 to 10 mM. Also number of supplementations is not particularly limited. For example, one to three times of supplementations may be conducted.

As described above, in the present invention, it is possible to suppress degradation of an mRNA only by adding pAp. Therefore, addition of an RNase inhibitor, or methods having conducted for suppressing degradation from the end of an mRNA, for example, adding of a CAP structure and preparation of a looped mRNA are no longer required. However, addition of an RNase inhibitor (for example, 1 U/μL to 10 U/μL in a reaction solution), and any method which can be conducted for suppressing degradation of an mRNA from its end are not excluded from the present invention.

As described already, it is believed that pAp is metabolized by a component contained in a cell extract liquid. As far as a yeast cell goes, it is known that pAp is converted to AMP by Hal2p, and Hal2p is inhibited by lithium ions (for example, see The EMBO Journal (1997) 16, 7184-7195).

In the present invention, lithium ion Li⁺ may be contained in the cell-free protein synthesis reaction solution. By containing lithium ion in the reaction solution, it is possible to obtain the effect of suppressing degradation of an mRNA which is one of causes of reducing the protein synthesis amount, more effectively with higher sustention. Alternatively, since the use amount of pAp can be reduced by using lithium ion, it is possible to obtain the mRNA degradation suppressing effect by pAp with lower cost. This effect can be obtained even when the cell extract liquid is not derived from a yeast cell. For example, the effect by lithium ion can be effectively obtained even when the cell extract liquid is derived from an insect cell.

The source of lithium ion is not particularly limited, and biochemically acceptable lithium salts may be used. As such a lithium salt, for example, lithium chloride, lithium acetate and the like may be recited.

The concentration of lithium ion is not particularly limited, however, the final concentration in a reaction solution may be, for example, 25 to 400 mM, and preferably 50 to 100 mM. By selecting the concentration from such a range, it is possible to obtain an mRNA degradation suppressing effect more effectively.

The reaction temperature in cell-free protein synthesis is usually 10° C. to 40° C., and preferably in the range of 15° C. to 30° C. This is because when the reaction temperature is less than 10° C., the protein synthesis speed tends to decrease, whereas when the reaction temperature is higher than 40° C., essential components tend to be denatured.

Cell-free protein synthesis in the present invention may be conducted by a batch method. The reaction time is not particularly limited, and may be appropriately selected by a person skilled in the art. For example, about 6 hours may be a reference.

The protein synthesized by the cell-free system protein synthesis method of the present invention may be quantified by measurement of activity of an enzyme, SDS-PAGE, immunoassay and the like.

The protein synthesizable by the cell-free system protein synthesis method of the present invention is not particularly limited.

The cell-free protein synthesis method of the present invention may be applied to a ribosome display method utilizing a cell-free protein synthesis system, or a in vitro molecule selecting method such as an in vitro virus method. The ribosome display method is a method that makes an mRNA and a protein generating by translation of the mRNA form a complex via a ribosome. As a reference to the ribosome display method, for example, Brief Funct Genomic Proteomic. July 2002; 1(2): 204-12. Ribosome display: cell-free protein display technology. He M, Taussig M J. may be recited. In the in vitro virus method, after puromycin is caused to bond on the 3′ end of an mRNA via poly(dA) or a PEG linker, a C-terminus of a protein generating as a result of translation and puromycin are allowed to react in a ribosome, to thereby form a protein-mRNA bonded molecule. An mRNA part of this bonded molecule is reverse-transcribed, and converted to double strand a nucleic acid of cDNA-mRNA. As a reference to the in vitro virus method, Miyamoto-Sato, E. et al: Genome res., 15, 710(2005) Cell-free co-translation and selection using in vitro virus for high-throughput analysis of protein-protein interactions and complexes may be recited.

As described above, in the method of the present invention, it is possible to readily suppress degradation of an mRNA in the cell-free protein synthesis reaction solution only by adding pAp. Therefore, the method of the present invention is more convenient and hence more practical than a cell-free protein synthesis method using a conventional mRNA degradation suppressing method.

### EXAMPLES

In the following, the present invention will be described in more detail by way of examples, however, the present invention is not limited to these examples.

### Example 1

### Step 1. Construction of Expressing Plasmid

PCR was conducted by using a human lysozyme cDNA clone (pERI 8602, Kanaya et al., J. Biol. Chem. 1992, 267, 15111-15115) as a template, and using primer sets having sequences respectively shown by the SEQ ID NO.: 1 and SEQ ID NO.: 2 below, and KOD-Plus-(TOYOBO).

| | |
|---|---|
| 5′-ATGAAGGTTTTCGAGAGATGCG-3′ | (SEQ ID NO.: 1) |
| 5′-GGGGTACCAACACCACAACCTTGAACG-3′ | (SEQ ID NO.: 2) |

The 5′-end of the DNA fragment amplified by PCR was phosphorylated by T4 Polynucleotide Kinase (TOYOBO), and digested by KpnI (TOYOBO). The resultant DNA was coupled to an EcoRV/KpnI site of pTD1 vector (SHIMADZU CORPORATION) by T4 ligase (Quick Ligation(™) Kit, Nebr.). A target plasmid derived from a clone obtained by transformation of E. coli DH5α was named pTD1-strep-h-LYZ (Ezure et al., Proteomics, in press).

### Step 2. In Vitro Transcription Reaction and Purification of mRNA

Using the expressing plasmid pTD1-strep-h-LYZ created in the above step 1 as a template, PCR was conducted by using primer sets having sequences respectively shown by the SEQ ID NO.: 3 and SEQ ID NO.: 4 below, and KOD-Plus-(TOYOBO).

| | |
|---|---|
| 5′-GCAGATTGTACTGAGAGTG-3′ | (SEQ ID NO.: 3) |
| 5′-GCGGATAACAATTTCACAC-3′ | (SEQ ID NO.: 4) |

The amplified fragment was purified by phenol-chloroform extraction and ethanol precipitation. Using 5 μg of the purified amplified fragment as a template, an mRNA was synthesized by a transcription reaction at 37° C. for 30 minutes in a scale of 100 μL using T7 RiboMAX(™) Express Large Scale RNA Production System (Promega). The obtained reaction solution was applied to Nick column (manufactured by Amersham Bioscience) and then eluted with pure water. Potassium acetate was added to the eluted fraction so that the final concentration was 0.3 M, and ethanol precipitation was conducted to purify the mRNA. The purified mRNA was quantified by measuring the absorbance at 260 nm and 280 nm.

### Step 3. Translation Reaction

Using Transdirect insect cell (SHIMADZU CORPORATION), each of the following reaction solutions was prepared in a scale of 50 μL. Transdirect insect cell is a cell-free protein synthesis reaction solution containing an extract from Sf21, and contains a potassium salt, a magnesium salt, adenosine triphosphate, guanosine triphosphate, creatine phosphate, creatine kinase, an amino acid component, a tRNA, a foreign mRNA, and a buffer as ingredients other than the extract derived from Sf21.

As the RNase inhibitor, an RNase inhibitor derived from a human placenta (TAKARA BIO) was used, and as the pAp, Adenosine 3′,5′-diphosphate sodium salt (SIGMA-ALDRICH) was used, and prepared as an aqueous solution of 100 mM. Each reaction solution was subjected to a translation reaction at 25° C.

Reaction Solution 1 (Test section for comparison)

| | |
|---|---|
| mRNA | not added |
| RNase inhibitor | not added |
| pAp | not added |

Reaction Solution 2 (Test section for comparison)

| | |
|---|---|
| mRNA | added (final concentration 320 μg/mL) |
| RNase inhibitor | not added |
| pAp | not added |

Reaction Solution 3 (Test section for comparison)

| | |
|---|---|
| mRNA | added (final concentration 320 μg/mL) |
| RNase inhibitor | added (final concentration 1 U/μL) |
| pAp | not added |

Reaction Solution 4 (Test section of present invention)

| | |
|---|---|
| mRNA | added (final concentration 320 μg/mL) |
| RNase inhibitor | not added |
| pAp | added (final concentration 5 mM) |

### Step 4. RNA Extraction

For each reaction solution, reaction solutions after 0, 60, and 120 minutes from starting of a translation reaction were recovered. 10 μL of the recovered reaction solution was collected into 20 μL of TRIzol LS Reagent (Invitrogen), and the whole RNA was extracted. The extracted RNA was dissolved in 10 μL of pure water.

### Step 5. Separation and Detection of RNA

To each solution of extracted RNA, 4 μL of a loading buffer (Wako Pure Chemical Industries) was added, and the total was subjected to electrophoresis (a TAE buffer, 1% agarose gel). The RNA separated by electrophoresis was detected by EtBr (ethidium bromide) staining.

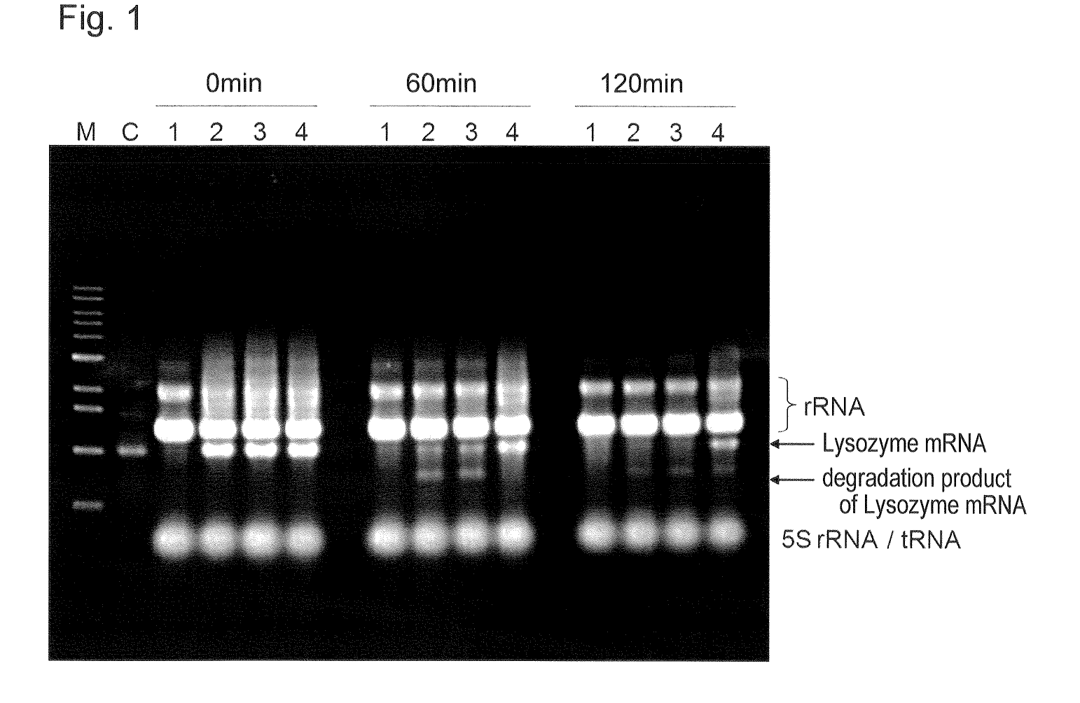

The obtained electrophoresis image is shown in FIG. 1. In FIG. 1, separation results of the whole RNA in the reaction solution at reaction times of 0, 60, and 120 minutes are shown in lane 1 for Reaction Solution 1 (mRNA non-added section), in lane 2 for Reaction Solution 2 (mRNA added section), in lane 3 for Reaction Solution 3 (mRNA and RNase inhibitor added section), and in lane 4 for Reaction Solution 4 (mRNA and pAp added section). Lane M shows an electrophoresis result for 1 kb ladder DNA size marker (Bioneer), and lane C shows an electrophoresis result of only an mRNA.

### Examination of mRNA Degradation Suppressing Effect

From the obtained electrophoresis results, mRNA remaining amounts in each test section and each reaction time was examined. Comparison between data of Reaction Solution 2 (mRNA added section) and data of Reaction Solution 3 (mRNA and RNase inhibitor added section) revealed that an mRNA degradation suppressing effect was not observed in a commercially available RNase inhibitor. On the other hand, from the data of Reaction Solution 4 (mRNA and pAp added section) at reaction times of 60 minutes and 120 minutes, it was found that pAp has an apparent degradation suppressing effect.

### Example 2

By the same steps as Steps 1 to 3 of Example 1, a translation reaction was conducted for the following reaction solutions.

Reaction Solution 4 (Test section of present invention):

| | |
|---|---|
| mRNA | added (final concentration 320 μg/mL) |
| RNase inhibitor | not added |
| pAp | added (addition before reaction: |
| | final concentration 5 mM, |
| | supplementation: not conducted) |

Reaction Solution 5 (Test section of present invention):

| | |
|---|---|
| mRNA | added (final concentration 320 μg/mL) |
| RNase inhibitor | not added |
| pAp | added (addition before reaction: |
| | final concentration 10 mM, |
| | supplementation: not conducted) |

Reaction Solution 6 (Test section of present invention):

| | |
|---|---|
| mRNA | added (final concentration 320 μg/mL) |
| RNase inhibitor | not added |
| pAp | added (addition before reaction: |
| | final concentration 5 mM, |
| | supplementation: conducted) |

As for Reaction Solution 6, pAp was added before the reaction so that the final concentration was 5 mM, and after 60 minutes from starting of the reaction, 2.5 μL of a 100 mM pAp aqueous solution prepared in step 3 was added. As a result, the pAp concentration of supplemented pAp excluding the pAp that was present from the initial stage of the reaction was 5 mM.

Steps 4 and 5 as same as those in Example 1 were conducted except that for each reaction solution, the reaction solution was recovered after 0, 120, 180, and 240 minutes from starting of the translation reaction.

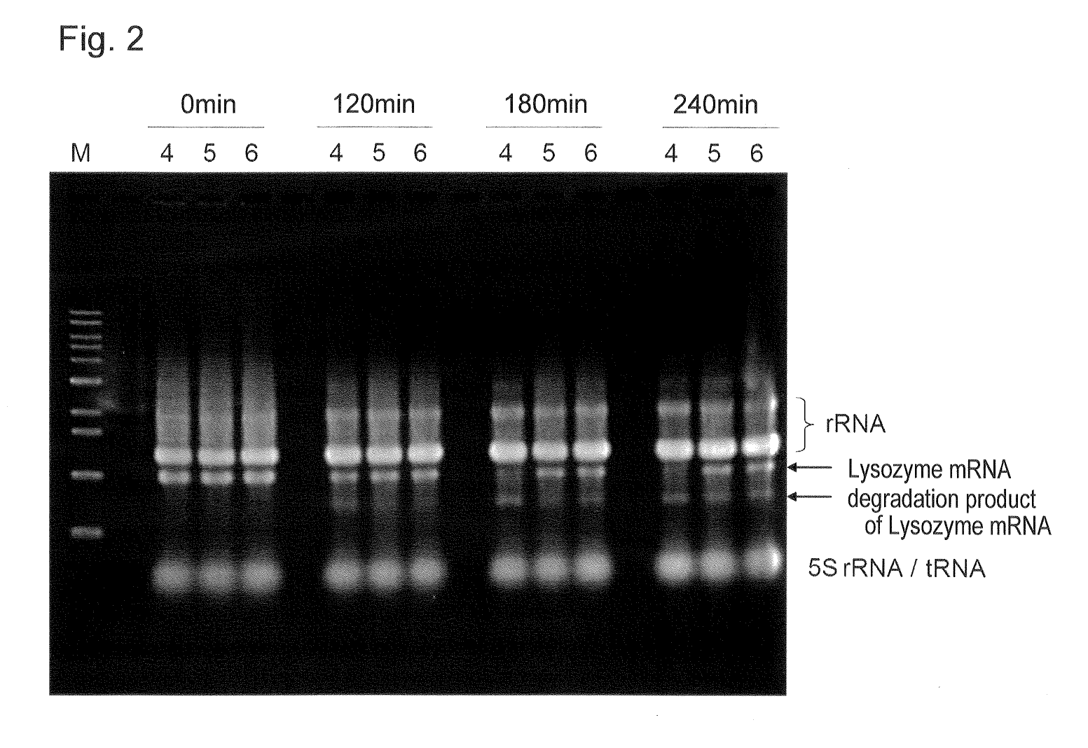

The obtained electrophoresis image is shown in FIG. 2. In FIG. 2, separation results of the whole RNA in the reaction solution at reaction times of 0, 120, 180, and 240 minutes are shown in lane 4 for Reaction Solution 4 (the section where pAp was added in a final concentration of 5 mM, and supplementation was not conducted), in lane 5 for Reaction Solution 5 (the section where pAp was added in a final concentration of 10 mM, and supplementation was not conducted), and in lane 6 for Reaction Solution 6 (the section where pAp was added in a final concentration of 5 mM, and pAp was supplemented after 60 minutes). Lane M shows an electrophoresis result for 1 kb ladder DNA size marker (Bioneer).

From the obtained electrophoresis results, mRNA remaining amounts in each test section and each reaction time was examined. Data of Reaction Solution 4 (the section where 5 mM pAp was added and supplementation was not conducted) after 180 minutes revealed that an RNA degradation suppressing effect of pAp was lost with time, while data of Reaction Solution 5 (the section where 10 mM pAp was added, and supplementation was not conducted) after 240 minutes and data of Reaction Solution 6 (the section where 5 mM pAp was added, and supplementation was conducted) after 240 minutes revealed that a degradation suppressing effect was sustained by increasing the addition amount of pAp or conducting supplementation.

From the results of Example 1 and Example 2, it was found that degradation of an mRNA can be suppressed by pAp added to a reaction solution and that the sustained time of the degradation suppressing effect can be extended depending on its addition amount.

### Example 3

By the same steps as Steps 1 to 3 of Example 1, a translation reaction was conducted for the following reaction solutions.

Reaction Solution 4 (Test section of present invention):

| | |
|---|---|
| mRNA | added (final concentration 320 μg/mL) |
| RNase inhibitor | not added |
| pAp | added (final concentration 5 mM) |
| LiCl | not added |

Reaction Solution 7 (Test section for comparison):

| | |
|---|---|
| mRNA | added (final concentration 320 μg/mL) |
| RNase inhibitor | not added |
| pAp | not added |
| LiCl | added (final concentration 50 mM) |

Reaction Solution 8 (Test section of present invention):

| | |
|---|---|
| mRNA | added (final concentration 320 μg/mL) |
| RNase inhibitor | not added |
| pAp | added (final concentration 5 mM) |
| LiCl | added (final concentration 50 mM) |

In Reaction Solutions 7 and 8, a 4M aqueous LiCl solution was prepared for stock, and using the stock solution, the final concentration in the reaction solution was adjusted to 50 mM.

Steps 4 and 5 were conducted in the same manner as in Example 1 except that for each reaction solution, the reaction solution was recovered after 0, 180, and 240 minutes from starting of the translation reaction.

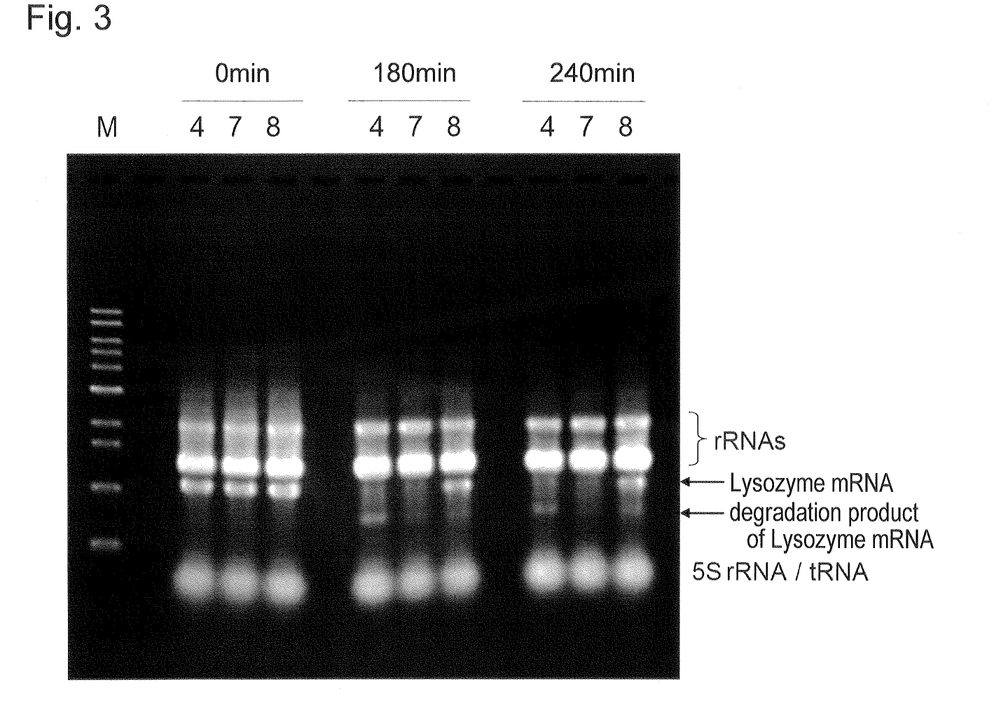

The obtained electrophoresis image is shown in FIG. 3. In FIG. 3, separation results of the whole RNA in the reaction solution at reaction times of 0, 180, and 240 minutes are shown in lane 4 for Reaction Solution 4 (the section where pAp was added in a final concentration of 5 mM, and LiCl was not added), in lane 7 for Reaction Solution 7 (the section where LiCl was added in a final concentration of 50 mM, and pAp was not added), and in lane 8 for Reaction Solution 8 (the section where pAp was added in a final concentration of 5 mM, and LiCl was added in a final concentration of 50 mM). Lane M shows an electrophoresis result for 1 kb ladder DNA size marker (Bioneer).

As shown in FIG. 3, in the sections where pAp and LiCl are respectively added singularly (lane 4 and lane 7), it was observed that almost all the mRNA was degraded after 180 minutes, however, in the section where both pAp and LiCl were added (lane 8), it was observed that the mRNA remained even after 240 minutes. This revealed that the degradation suppressing effect is sustained by adding LiCl in addition to pAp to a reaction solution.

In the above examples, concrete embodiments within the scope of the present invention have been shown, however, the present invention may be carried out in various other embodiments without limited thereto. Therefore, the above examples are given for illustration in every point, and should not be interpreted in a limitative manner. Furthermore, all modifications within equivalents of claims are involved in the scope of the present invention.

What is claimed is:

1. A cell-free protein synthesis method using a cell-free protein synthesis reaction solution containing at least an extract liquid derived from a living cell, a potassium salt, a magnesium salt, adenosine triphosphate, guanosine triphosphate, creatine phosphate, creatine kinase, amino acid, a tRNA, an mRNA, a buffer, and adenosine 3′,5′-bisphosphate.

2. The cell-free protein synthesis method according to claim 1, wherein the cell-free protein synthesis reaction solution further contains lithium ion Li⁺.

3. The cell-free protein synthesis method according to claim 1, wherein while the cell-free protein synthesis reaction is sustained, adenosine 3′,5′-bisphosphate is supplemented to the cell-free protein synthesis reaction solution.

4. The cell-free protein synthesis method according to claim 1, wherein the living cell is an insect culture cell.

5. A cell-free protein synthesis reaction solution containing at least an extract liquid derived from a living cell, a potassium salt, a magnesium salt, adenoslne triphosphate, guanosine triphosphate, creatine phosphate, creatine kinase, amino acid, a tRNA, an mRNA, a buffer, and adenosine 3′,5!-bisphosphate.

6. The cell-free protein synthesis reaction solution according to claim 5, further containing lithium ion Li⁺.
